(19) United States
(12) Patent Application Publication
(10) Pub. No.: US 2011/0054487 A1
(43) Pub. Date: Mar. 3, 2011

(54) COAXIAL TRANSSEPTAL GUIDE-WIRE AND NEEDLE ASSEMBLY

(75) Inventor: Robert C. Farnan, Rivervale, NJ (US)

(73) Assignee: CIRCULITE, INC., Saddle Brook, NJ (US)

(21) Appl. No.: 12/794,847

(22) Filed: Jun. 7, 2010

Related U.S. Application Data
(60) Provisional application No. 61/239,151, filed on Sep. 2, 2009.

(51) Int. Cl. A61M 25/09; A61B 17/34

(52) U.S. Cl. 606/108; 606/185

(57) ABSTRACT

A coaxial transseptal device and methods of piercing a tissue within the heart. The coaxial transseptal device includes a piercing device having a shaft with a distal sharpened portion. The coaxial transseptal device also includes a coaxial guide-wire configured to receive the piercing device and move relative thereto. The flexibility of the coaxial guide-wire increases from the proximal end to the distal end.

### CROSS-REFERENCE TO RELATED APPLICATION

This application claims the priority of U.S. Provisional Patent Application Ser. No. 61/239,151, filed on Sep. 2, 2009 (pending), the disclosure of which is incorporated by reference herein.

### TECHNICAL FIELD

The present invention generally relates to devices and methods of crossing a tissue and, more particularly, devices and methods of percutaneously piercing through an internal tissue of the heart.

### BACKGROUND

The circulatory system of the human body transports blood containing chemicals, such as metabolites and hormones, and cellular waste products to and from the cells. This organ system includes the heart, blood, and a vascular network. Veins are vessels that carry blood toward the heart while arteries carry blood away from the heart. A septum separates the left and right sides of the heart where each side includes an atrial chamber and a ventricular chamber. The atrial chambers receive blood from the veins and the ventricular chambers, which include larger muscular walls, pump blood from the heart. Movement of the blood is as follows: blood enters the right atrium from either the superior or inferior vena cava and moves into the right ventricle. From the right ventricle, blood is pumped to the lungs via pulmonary arteries to become oxygenated. Once the blood has been oxygenated, the blood returns to the heart by entering the left atrium, via the pulmonary veins, and flows into the left ventricle. Finally, the blood is pumped from the left ventricle into the aorta and the vascular network.

A number of surgical procedures are performed on the internal tissues of the heart, such as the implanting of a cardiac assist devices for treating congenital heart disease or valve procedures for repairing a prolapsing valve. Conventionally these procedures involved a thoracotomy, i.e., the opening of the thoracic cavity between successive ribs to expose the internal organs. More typical is cardiac surgery, generally known as open-heart surgery, where the sternum is cut and split to expose the internal organs. Once the thoracic cavity is accessed, the physician must enter the pleural space and puncture both the pericardium and the myocardial wall. There are great risks and an extensive recovery time associated with the invasive nature of the implantation surgery. As such, some patients with severe symptoms are not healthy enough for surgery to receive a circulatory assist system.

There have been some catheter-based procedures developed for accessing the chambers of the heart. Conventionally these procedures are performed from a vascular access site near the right femoral vein in order to accommodate the angle between the vena cava and the septum. Yet, there continues to be a need for improvements in crossing the septum and treating defects associated with the atrial septum (e.g., ASDs, PFOs).

### SUMMARY OF THE INVENTION

In one illustrative embodiment of the present invention, a coaxial transseptal device for piercing a tissue within the heart is described. The coaxial transseptal device includes a piercing device with a shaft and a distal needle portion. The coaxial transseptal device also includes a coaxial guide-wire configured to receive the piercing device and move relative thereto and has a flexibility that increases distally.

In another illustrative embodiment a guide-wire is described. The guide-wire comprises a tube with proximal and distal ends and a lumen extending between. The flexibility of the tube increases distally. A coil surrounds at least the distal end of the tube and a hub is attached to the proximal end of the tube.

Another illustrative embodiment describes a piercing device that includes a shaft having a needle portion on the distal end. The flexibility of the needle portion increases distally.

In yet another illustrative embodiment of the present invention, a method of piercing a tissue within the heart of a patient with the coaxial transseptal device is provided. The method includes introducing the distal end of the coaxial guide-wire into a superficial blood vessel. The distal end of the coaxial guide-wire is then directed through the superficial blood vessel and to the tissue within a first chamber of the heart. The needle portion of the piercing device is advanced beyond the distal end of the coaxial guide-wire, across the tissue, and into a second chamber of the heart. The distal end of the coaxial guide-wire is then advanced over the piercing device, across the tissue, and into the second chamber, which dilates the puncture in the tissue.

The guide-wire can include a removable adapter on the proximal end that can couple to a pressure monitor. The removable adapter and pressure monitor are configured to determine a pressure within a chamber of the heart.

### BRIEF DESCRIPTION OF THE FIGURES

FIGS. 1A and 1B are diagrammatic views of an exemplary method of accessing the septum of the human heart with a coaxial transseptal guide-wire and needle assembly, shown in cross-section.

FIG. 2A is a disassembled, side-elevational view of the coaxial transseptal guide-wire and needle assembly.

FIG. 2B is an assembled, side-elevational view of the distal end of the coaxial transseptal guide-wire and needle assembly.

FIG. 2C is an assembled, side-elevational view of the proximal end of the coaxial transseptal guide-wire and needle assembly with a pressure adaptor.

FIG. 3A is an assembled, side-elevational view of the distal end of the coaxial transseptal guide-wire and needle assembly, shown in partial cross-section.

FIG. 3B is an assembled, side-elevational view of an alternate embodiment of the distal end of the coaxial transseptal guide-wire and needle assembly, shown in partial cross-section.

FIGS. 4A-4B are side-elevational views of alternate embodiments of the distal end of a piercing device.

FIG. 4C is a cross-sectional view illustrating an alternate embodiment of the piercing device.

FIG. 5 is an assembled, side-elevational view of the coaxial transseptal guide-wire and needle assembly with a safety clip secured to the piercing device.

FIG. 6 is an assembled, side-elevational view of the coaxial transseptal guide-wire and needle assembly with the safety clip removed.

FIGS. 7A-7D are side-elevational views in partial cross section illustrating successive steps of one exemplary procedure for crossing the intra-atrial septum with the coaxial transseptal guide-wire and needle assembly.

FIG. 8 is a diagrammatic view of an alternate method of accessing the septum of the human heart with a coaxial transseptal guide-wire and needle assembly, shown in cross-section.

### DETAILED DESCRIPTION

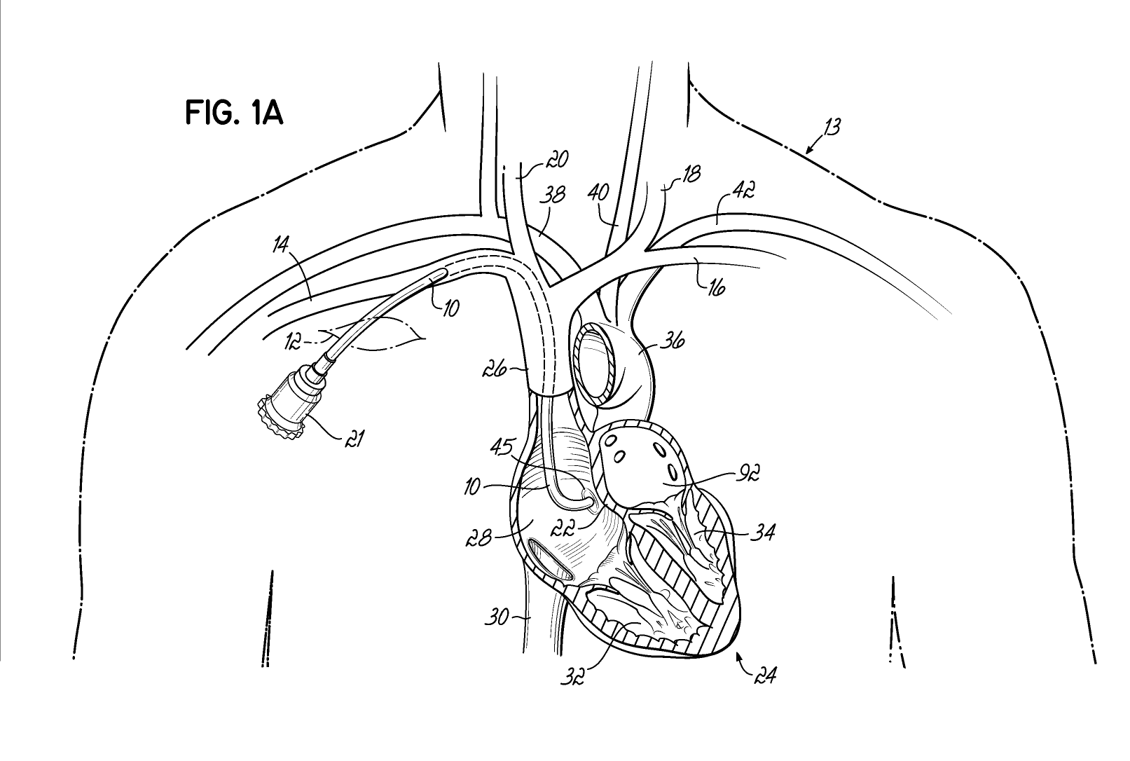

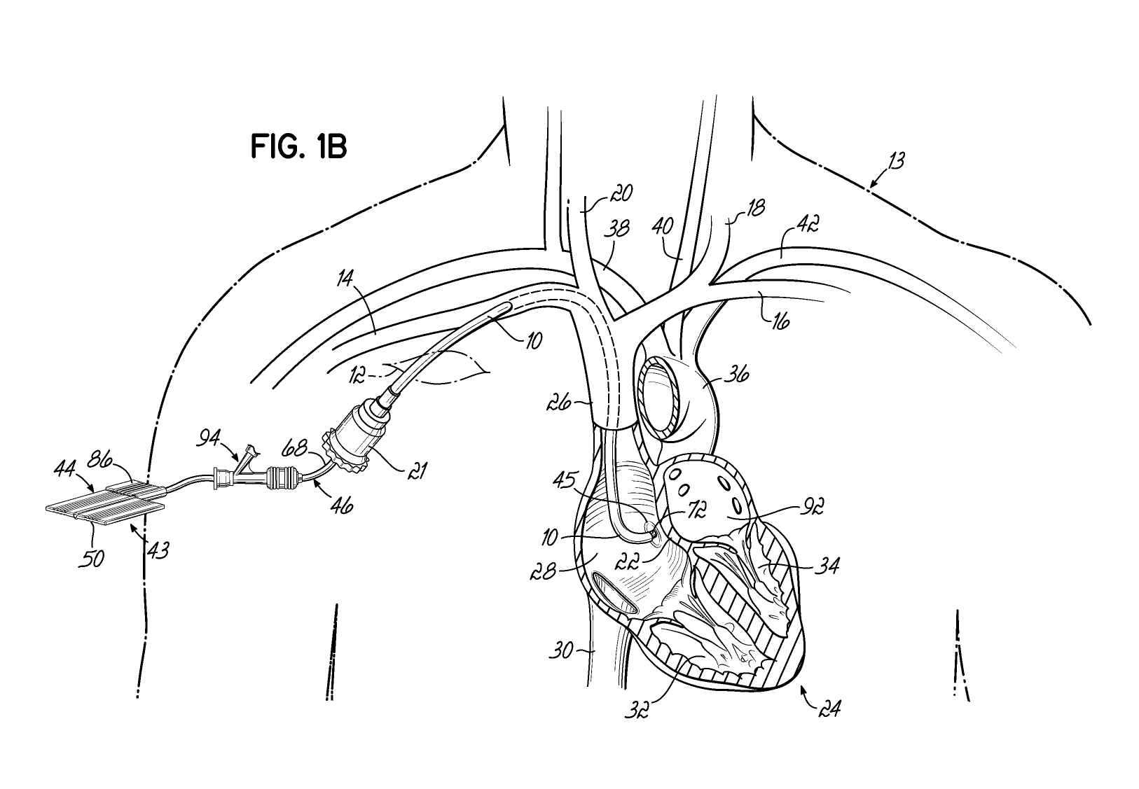

FIGS. 1A and 1B illustrate an exemplary method of transseptal crossing according to one embodiment of the present invention. Accordingly, the physician can direct a guide catheter 10 into a vascular access site 12 of the patient 13. The guide catheter 10 can be any steerable or preformed catheter that can be directed through the vascular system to aid in the delivery of subsequent surgical devices to the surgical site. The vascular access site can be near a suitable superficial blood vessel, such as the right subclavian vein 14; however, other superficial vessels could also be used, such as the left subclavian vein 16 or the left or right jugular veins 18, 20, or in some instances a superficial artery could also be used.

The guide catheter 10 can include a hub 21 having a hemostasis valve that prevents the loss of blood while maintaining access for passage of the subsequent devices.

The guide catheter 10 is directed to the intra-atrial septum 22 of the heart 24 via the superior vena cava 26 and the right atrium 28. For illustrative purposes additional anatomy is shown, including the inferior vena cava 30, the right ventricle 32, the left ventricle 34, the aortic arch 36, the brachiocephalic trunk 38, the left common carotid artery 40, and the left subclavian artery 42.

In FIG. 1B the distal end of the coaxial transseptal device 43 is directed into the guide catheter 10 at the vascular access site 12 and advanced through the lumen of the guide catheter 10 to the right atrium 28. Typically, the coaxial transseptal device 43 is advanced to an area of the right atrium 28 that is near the fossa ovalis 45.

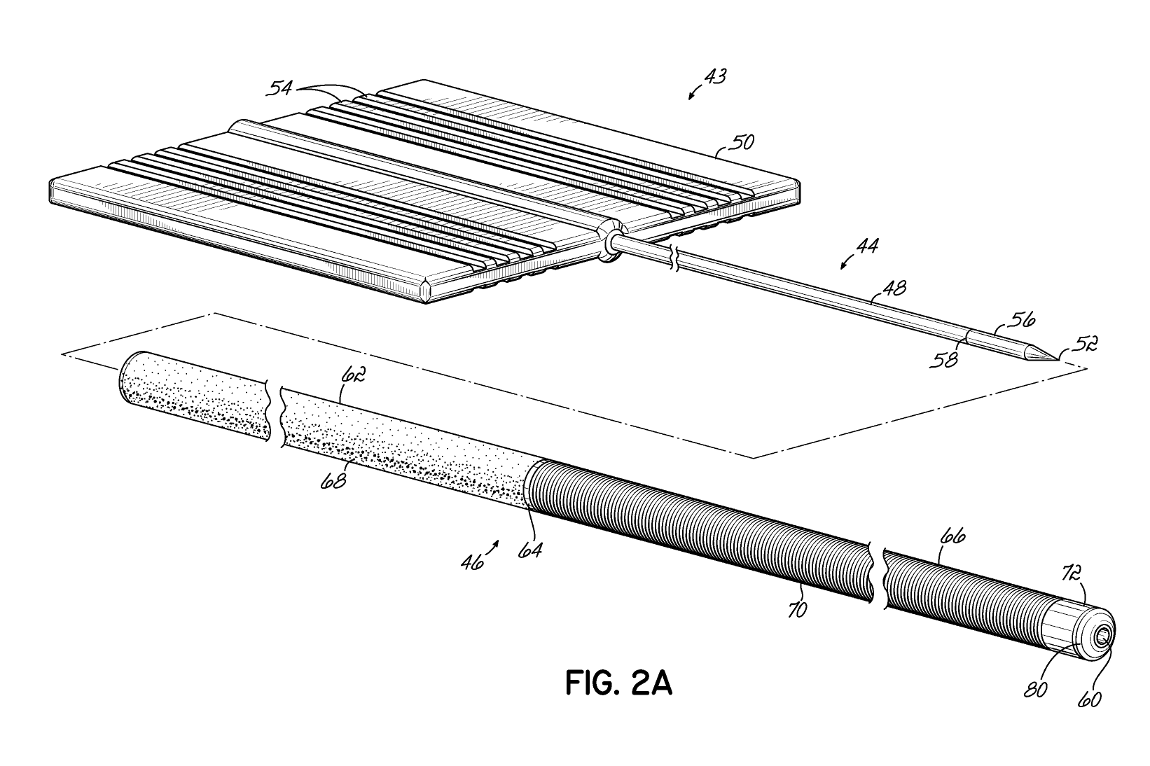

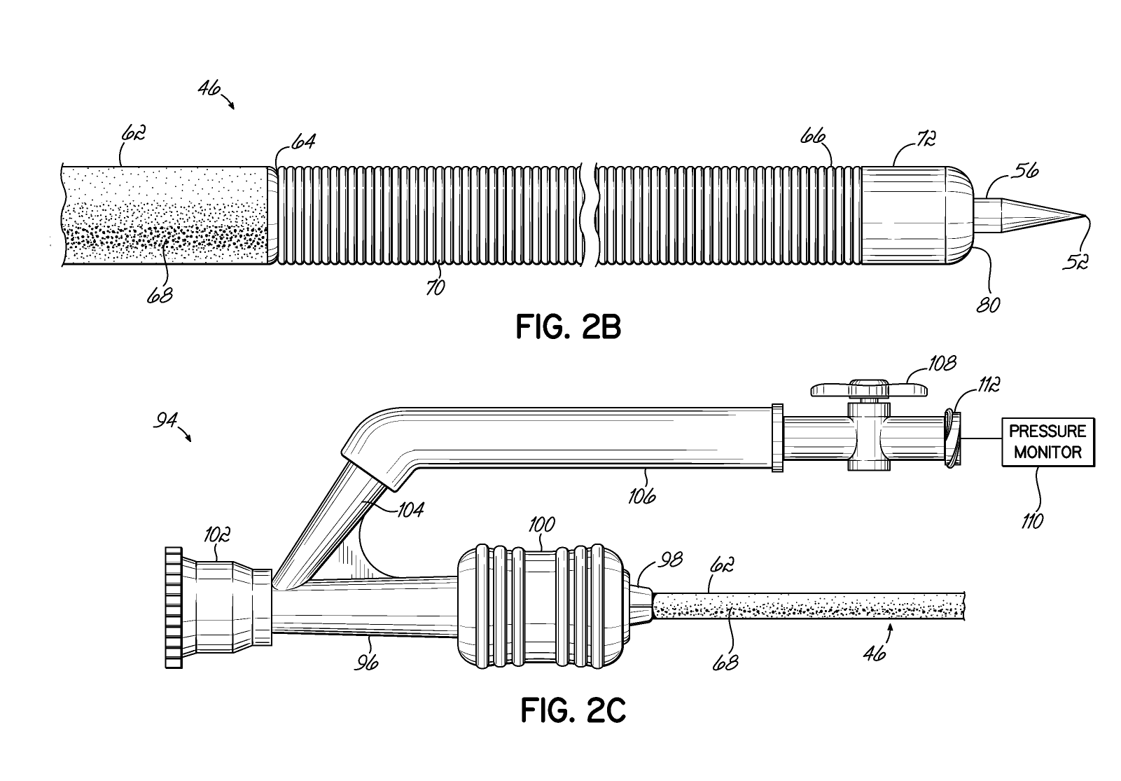

FIGS. 2A and 2B illustrate the details of the coaxial transseptal device 43, which includes a piercing device 44 and a coaxial guide-wire 46. The coaxial guide-wire 46 receives, and moves relative to, the piercing device 44.

As shown in FIG. 2A, the piercing device 44 includes a shaft 48 having distal and proximal ends, where the proximal end includes a hub 50 and the distal end includes a sharpened portion, i.e., a needle portion 52. The hub 50 can be secured to the flexible shaft 48 by either an insert injection molding process or by bonding a previously molded hub 50 to the shaft 48 using a biocompatible adhesive or epoxy. The hub 50 can include grooves 54 for providing additional grip between the physician's glove and the hub 50.

Referring still to FIG. 2A, the distal end of the shaft 48 can further include a radiopaque tip 56 constructed from any radiopaque material, such as platinum-iridium (Ptlr), stainless steel, tungsten (W), or tantalum (Ta). Radiopaque materials allow the physician to remotely visualize the structure, in vivo, by X-ray or real-time fluoroscopy. The distal end of the radiopaque tip 56 can be ground to an optimal shape for the needle portion 52, which facilitates the puncture and passage through a vascular tissue, such as the intra-atrial septum 22 (FIG. 1).

The shaft 48 can be constructed from metallic materials, such as MP35N, nickel titanium (NiTi), or stainless steel, or from a rigid polymer such as polyamide or polyaryletheretherketone (PEEK). To ensure visual contrast, the shaft material should not be as radiopaque as the material comprising the radiopaque tip 56. The joint 58 between the shaft 48 and the radiopaque tip 56 can be made by common welding techniques or by using a biocompatible adhesive or epoxy. The shaft 48 can also be coated with a lubricious polymer material to minimize friction between the flexible shaft 48 and the coaxial guide-wire 46. The shaft 48 is constructed with an outer diameter that is sized such that there is sufficient clearance between the outer diameter of the shaft 48 and the diameter of a lumen 60 extending through the coaxial guide-wire 46 to allow the shaft 48 to move relative to the coaxial guide-wire 46.

FIGS. 2A and 2B further illustrate the details of the coaxial guide-wire 46. The coaxial guide-wire 46 includes a proximal portion 62 that is separated by a transition joint 64 from a distal portion 66. The proximal portion 62 can include a proximal sleeve 68 constructed from a polymeric thermoset material, such as polytetrafluoroethylene (PTFE) or polyimide, or a thermoplastic polymer, such as fluorinated ethylene propylene (FEP), polyurethane, or polyamide. Generally, the material should have a low coefficient of friction or be a material that will accept a lubricious polymer material. While the thickness of the proximal sleeve 68 can vary and depend on a desired final outer diameter of the coaxial guide-wire 46, the wall thickness of the proximal sleeve 68 can vary from about 0.0127 mm (0.0005 inches) to about 3.81 mm (0.15 inches). Typical outer diameters for the guide-wire can range from about 0.012 inches to about 0.038 inches.

The coaxial guide-wire 46 can vary in length to accommodate various surgical procedures, but generally range from about 50 cm to about 400 cm.

The distal portion 66 of the coaxial guide-wire 46 can include a coil 70 extending between a distal tip 72 and the proximal sleeve 68. The coil 70 can be constructed from a metallic material, such as stainless steel or Ptlr, and is typically round in cross-section, though rectangular or flat wire cross-sections are possible. The round cross-section coils can range in diameter from about 0.0254 mm (0.001 inches) to about 0.254 mm (0.010 inches); flat wire coils can have a thickness-to-width ratio ranging from about 1:2 to about 1:4 with thickness ranging from about 0.0127 mm (0.0005 inches) to about 0.127 mm (0.005 inches). The coil 70 can be coated with a lubricious material, such as PTFE. In some embodiments, though not shown here, the coil 70 can extend the full length of the coaxial guide-wire. The proximal sleeve 68 and coil 70 have similar outer diameters to ensure a smooth transition between components and are joined at the transition joint 64 by common welding techniques or by using a biocompatible adhesive or epoxy.

The distal tip 72 can be constructed from a dense metal to enhance its radiopacity and has an outer diameter that is substantially similar to the outer diameter of the coil 70 to ensure a smooth transition between the components.

FIG. 2C illustrates a removable adapter 94 that can be attached to the proximal end of the coaxial guide-wire 46. The removable adapter 94 includes a body 96 and a distal collet 98 with an adjustment mechanism 100 for tightening the collet 98 against the coaxial guide-wire 46. The collet 98 can include an internal ring (not shown) for creating a fluid tight seal against the coaxial guide-wire 46. The proximal end of the body 96 can include a hemostasis valve hub 102 to prevent the loss of blood while maintaining a fluidic access to the lumen 60 (FIG. 2A) of the coaxial guide-wire 46. The body 96 further includes a side port 104 with tubing 106 and a stopcock 108, which can then be attached to a pressure monitor 110. The pressure monitor 110 can allow the physician to monitor the pressure within the left atrium 92 (FIG. 1A) during the surgical procedure, particularly when puncturing the intra-atrial septum 22 (FIG. 1A). By monitoring the pressure within the left atrium 92 (FIG. 1A) the physician can ensure that the transseptal crossing has occurred in the appropriate location. A luer fitting 112 can be used for attaching the tubing 106 to the pressure monitor 110 or other device within the operating room.

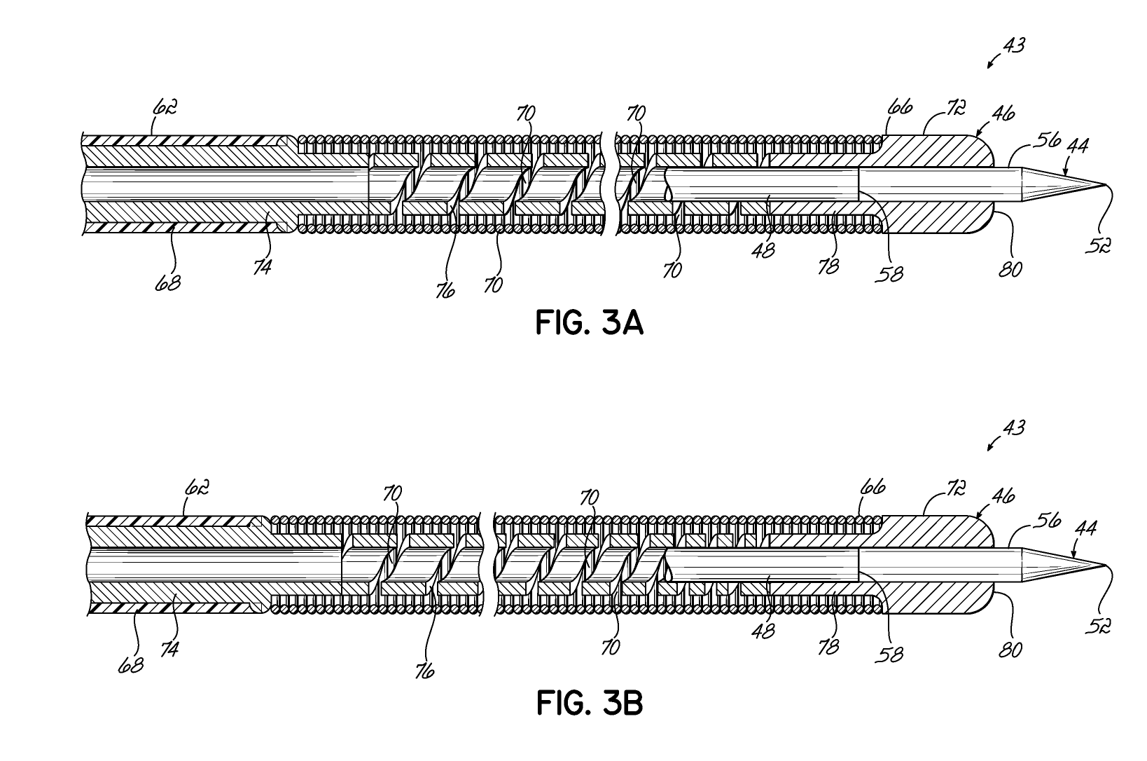

FIG. 3A illustrates the cross-sectional features of the distal ends of the coaxial guide-wire 46 and the piercing device 44. The coaxial guide-wire 46 includes a tube 74 extending proximally from the distal tip 72. Construction of the tube 74 can include metallic materials, such as MP35N, NiTi, or stainless steel. The tube 74 is formed by a wire drawing process and electro-polished to remove sharp edges. While the wall thickness of the tube 74 can vary, typical thicknesses can range from about 0.0254 mm (0.001 inches) to about 0.254 mm (0.010 inches).

The distal portion of the tube 74 can be processed by laser into a spiral cut section 76 to provide a flexibility that increases distally. That is, the distal portions of the spiral cut section 76 are more flexible than the proximal portions of the spiral cut section 76. As shown in FIG. 3A, the spiral cut section 76 can have a uniform pitch at the distal end of the tube 74. Alternatively, as shown in FIG. 3B, the spiral cut section 76 can have a variable pitch such that the flexibility of the spiral cut section is further increased distally, i.e., the proximal section of the spiral cut section 76 has a larger pitch than the more flexible, distal section. The spiral cut section 76 allows the distal end of the coaxial guide-wire 46 to be flexible enough to pass through the vascular system while still limiting the radius of curvature. The spiral cut section 76 should be constructed with a direction that opposes the direction of the pitch of the coil 70 to prevent the coil 70 from penetrating into the spiral cut section 76 as the coaxial guide-wire 46 is passed through a bend in the vascular system.

The distal tip 72 includes a tip shoulder 78 and a radial tip 80. The tip shoulder 78 provides a surface for adjoining the distal tip 72, the coil 70, and the tube 74 by common welding techniques or by using a biocompatible adhesive or epoxy. The radial tip 80 minimizes unintentional trauma to vascular tissue as the coaxial guide-wire 46 is advanced through the vascular network. While a rounded shaped radial tip 80 is shown, it is possible for the radial tip 80 to alternatively include a bullet shape, a bevel, or an elliptical shape.

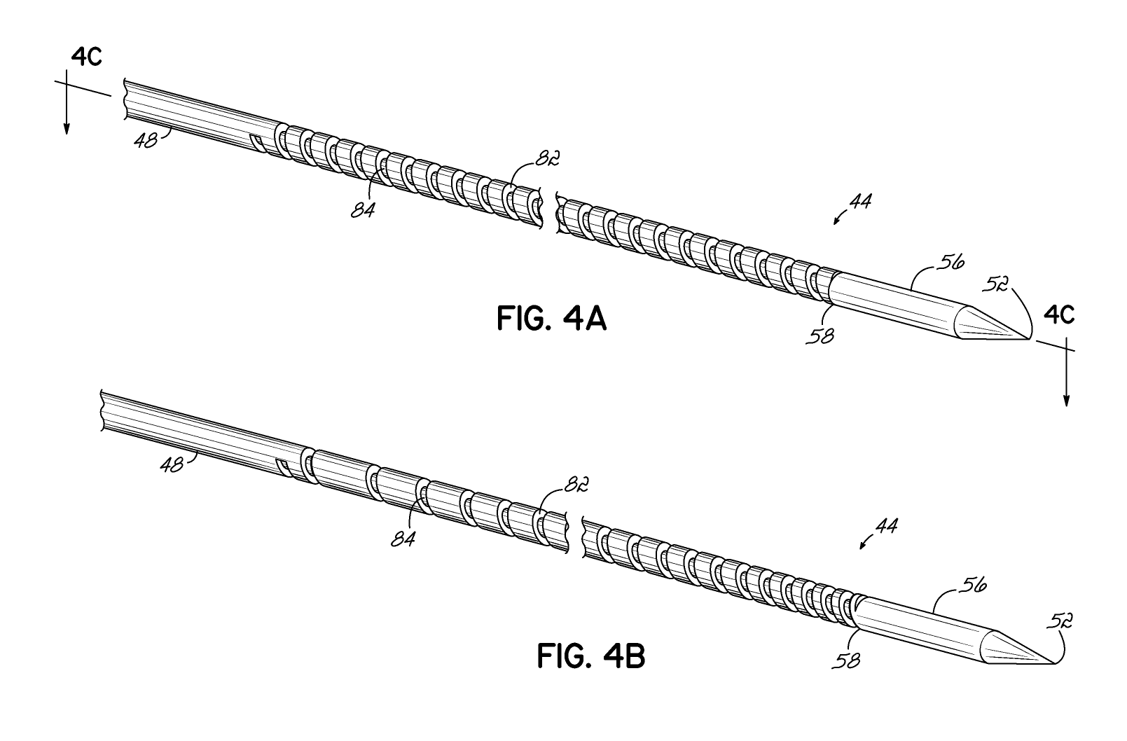

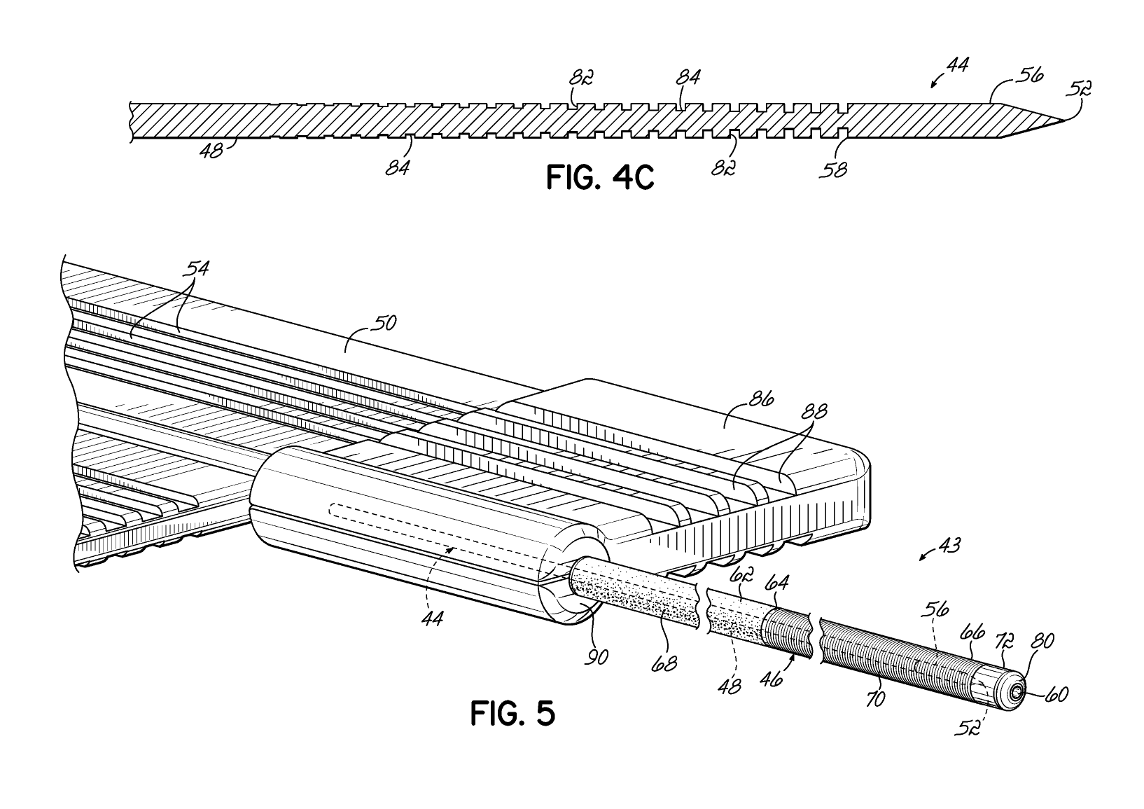

Turning now to FIG. 4A, one embodiment of the piercing device 44 is illustrated. As shown, the flexible shaft 48 can include a distally-positioned spiral cut section 82, which can be made by laser machining. The spiral cut section 82 is typically helical and can penetrate into the material of the shaft 48 by no more than ½ of the original outer diameter of the shaft 48. This would allow at least ½ of the original outer diameter to remain as a core 84. The core 84 provides structural stability to aid in advancing the piercing device 44 through the coaxial guide-wire 46. The spiral cut section 82 provides flexibility to the distal end of the piercing device 44 as it advances through the coaxial guide-wire 46 within the vascular network.

FIGS. 4B and 4C illustrate alternate embodiments of the piercing device 44. In FIG. 4B, the spiral cut section 82 is cut with a variable pitch, as compared to the constant pitch of FIG. 4A. Accordingly, the variable pitch is such that the proximal portion of the spiral cut section 82 of the piercing device 44 has a greater pitch and is less flexible than the distal portion of the spiral cut section 82. FIG. 4C illustrates an embodiment where the spiral cut section 82 is cut with a variable depth such that the distal portion of the spiral cut section 82 is cut deeper and is therefore more flexible than the proximal portion. At the most distal portion of the spiral cut section 82, the helical cut should not penetrate into the material of the shaft 48 by more than ½ of the original outer diameter.

FIG. 5 illustrates the assembled coaxial transseptal device 43. The piercing device 44 is back-loaded into the coaxial guide-wire 46 until the hub 50 is positioned near the proximal end of the coaxial guide-wire 46. To ensure that the needle portion 52 remains sheathed within the coaxial guide-wire 46, and to prevent inadvertent and premature puncture of the vascular tissue, a safety clip 86 can be positioned on the shaft 48 between the hub 50 and the proximal end of the coaxial guide-wire 46. The safety clip 86 can be machined or molded from a thermoplastic material, a polymer, or metal and can include grooves 88 to improve the grip between the physician's glove and the safety clip 86. In the illustrative embodiment, the safety clip 86 is constructed with an attachment portion 90 that snaps onto the flexible shaft 48, though additional safety locks and features could also be used. The attachment portion 90 creates a frictional fit against the shaft 48 and prevents the needle portion 52 from prematurely advancing beyond the distal end of the coaxial guide-wire 46. The length of the attachment portion 90 that contacts the shaft 48 will determine the penetration depth of the piercing device 44 into the left atrium 92 (FIG. 1A), in a manner that is described in greater detail below.

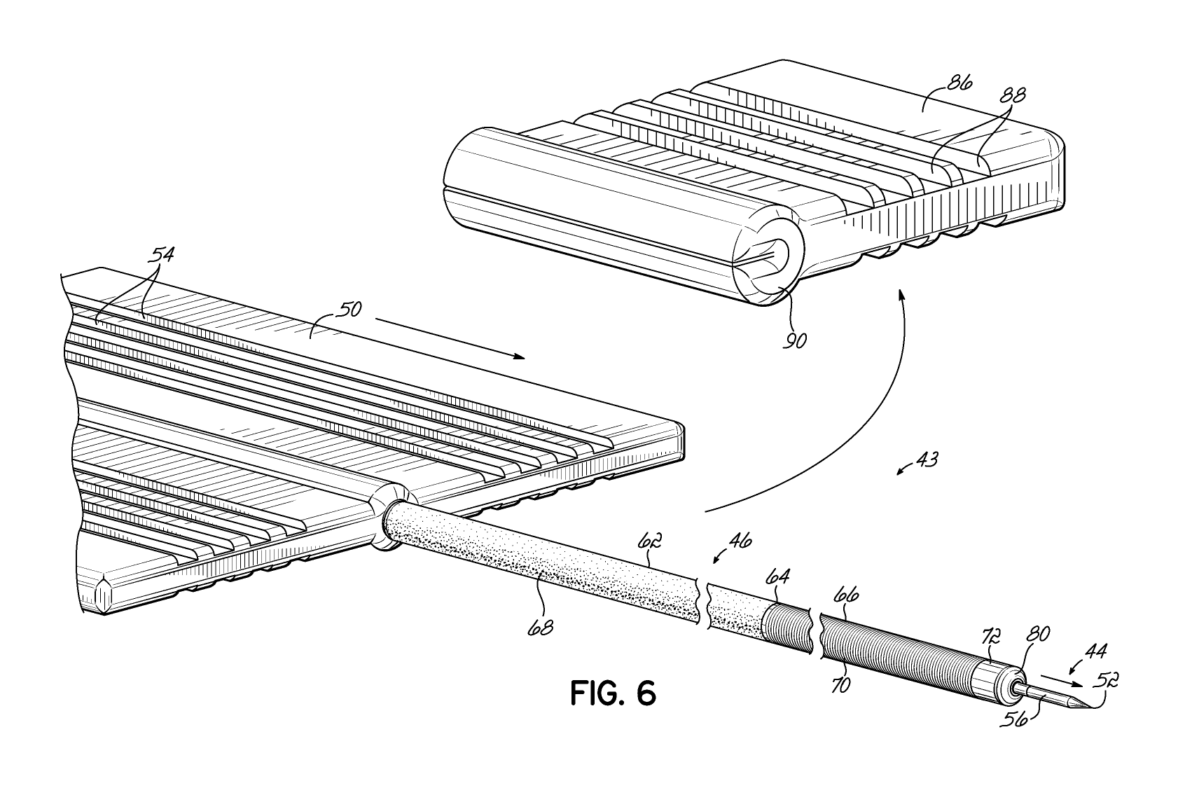

FIG. 6 illustrates the removal of the safety clip 86, which then allows the hub 50 of the piercing device 44 to advance distally and contact the proximal end of the coaxial guide-wire 46. As the hub 50 is advanced, the needle portion 52 extends distally from the radial tip 80 of the distal tip 72. Though not drawn to scale, from FIG. 6 is can be seen that the piercing device 44 can only extend distally from the radial tip 80 by an amount that is equal to the length of the attachment portion 90 that contacted the shaft 48 in FIG. 5.

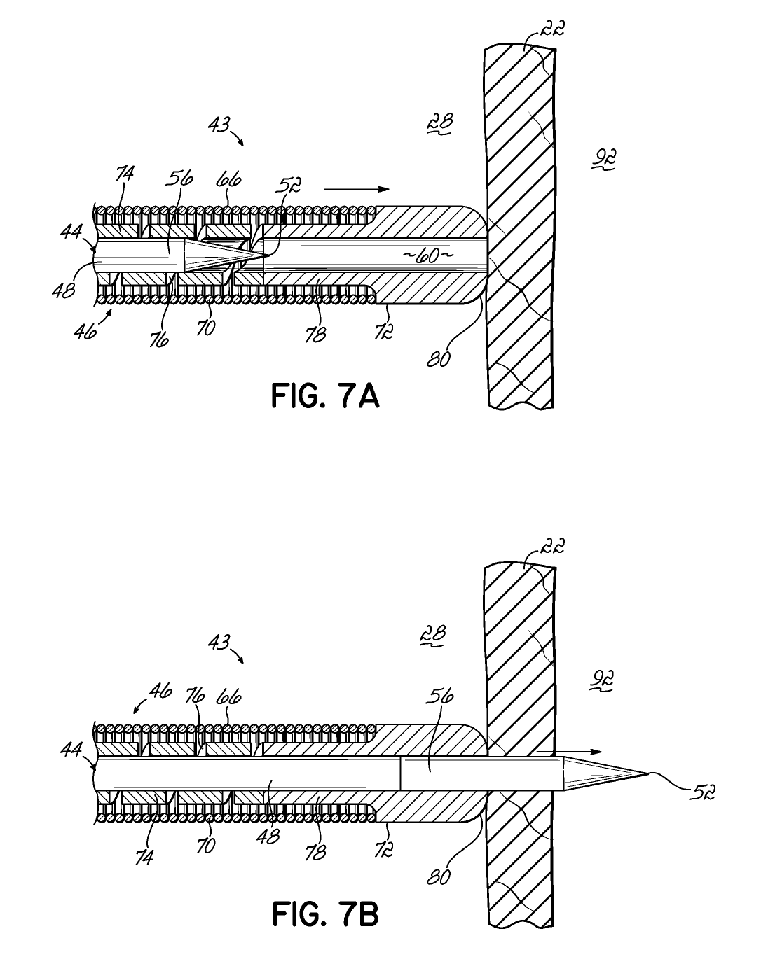

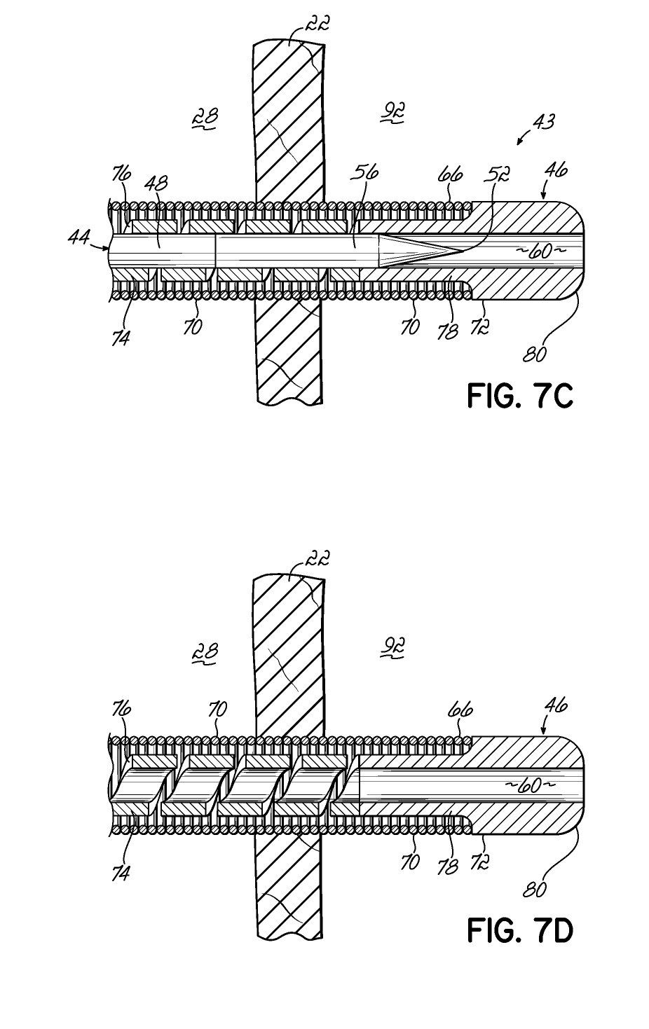

With the details of the coaxial transseptal device 43 described with some detail, one method of transseptal crossing can be described with reference to FIGS. 7A-7D.

FIG. 7A illustrates the coaxial transseptal device 43 as it is advanced into the right atrium 28 and such that the radial tip 80 contacts the intra-atrial septum 22. The needle portion 52 remains sheathed within the coaxial guide-wire 46.

In FIG. 7B, the physician can remove the safety clip 86 (FIGS. 5 and 6), if used, to release the shaft 48 such that it is distally moveable with respect to the coaxial guide-wire 46. The physician can then advance the piercing device 44, as had been shown previously in FIGS. 5 and 6, such that the hub 50 is advanced toward the proximal end of the coaxial guide-wire 46. Coincidentally, the needle portion 52 extends beyond the distal end of the radial tip 80 and punctures the intra-atrial septum 22. Continued advancement of the piercing device 44 causes the needle portion 52 to pass across the intra-atrial septum 22 and into the volume of the left atrium 92. During the piercing and crossing of the intra-atrial septum 22, the physician can constantly monitor the pressure within the left atrium with a pressure monitor via the removable adapter 94 (FIG. 2C).

In FIG. 7C, the physician advances the coaxial guide-wire 46 across the intra-atrial septum 22 while maintaining the relative position of the piercing device 44 to the coaxial guide-wire 46 so as to not puncture additional tissues. The radial tip 80 can be used to dilate the puncture created by the piercing device 44 through the intra-atrial septum 22 to a diameter that is similar to the outer diameter of the coaxial guide-wire 46.

With the distal tip 72 of the coaxial guide-wire 46 in the left atrium 92, the physician can then retract the piercing device 44 from the coaxial guide-wire 46. The removable adapter 94 (FIG. 2C) is removed leaving only the coaxial guide-wire 46 in place and as shown in FIG. 7D. The coaxial guide-wire 46 is then prepared to receive auxiliary devices.

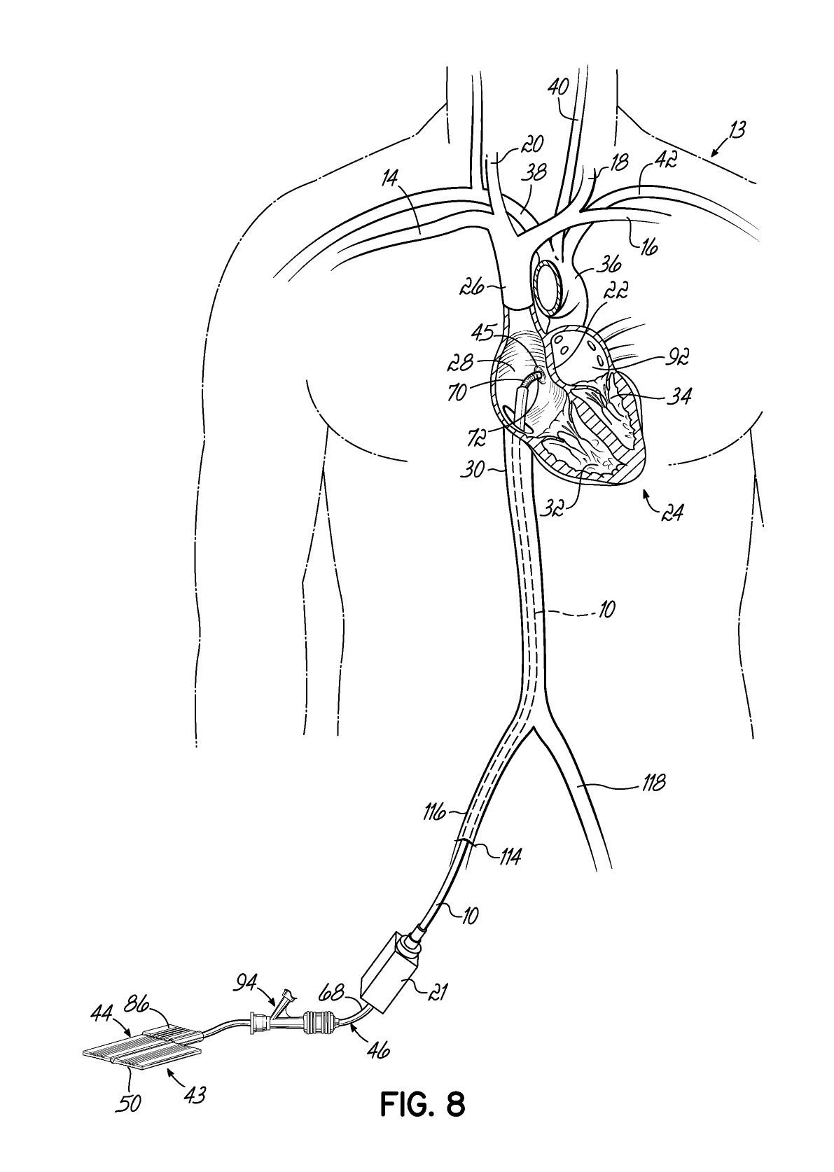

FIG. 8 illustrates an alternate method of accessing a tissue within the heart 24 of a patient 13. As shown, a vascular access site 114 can be chosen to be from an inferior location, such as the right or left femoral veins 116, 118. The guide catheter 10 and coaxial transseptal device 43 are then directed to the right atrium 28 from the inferior vena cava 30. The physician can then cross the intra-atrial septum 22 in a manner similar to the procedure described in detail above.

While the present invention has been illustrated by a description of various preferred embodiments and while these embodiments have been described in some detail, it is not the intention of the Applicants to restrict or in any way limit the scope of the appended claims to such detail. Additional advantages and modifications will readily appear to those skilled in the art. The various features of the invention may be used alone or in any combination depending on the needs and preferences of the user. This has been a description of the present invention, along with the preferred methods of practicing the present invention as currently known. However, the invention itself should only be defined by the appended claims.

What is claimed is:

1. A coaxial transseptal device comprising:
a piercing device comprising a shaft having a proximal end and a distal end, wherein a sharpened portion is included on the distal end; and
a coaxial guide-wire configured to receive the piercing device and move relative thereto, the coaxial guide-wire having a proximal end and a distal end, wherein a flexibility of the coaxial guide-wire increases distally.

2. The coaxial transseptal device of claim 1, wherein the coaxial guide-wire includes a tube having a helical cut on a distal end of the tube for increasing the flexibility of the coaxial guide-wire.

3. The coaxial transseptal device of claim 2, wherein the helical cut has a pitch that varies along the tube, whereby the pitch is smaller at a distal end than at a proximal end of the helical cut.

4. The coaxial transseptal device of claim 2, wherein the coaxial guide-wire includes a coil surrounding at least the distal portion of the tube.

5. The coaxial transseptal device of claim 4, wherein a direction of the helical cut opposes a direction of the coil.

6. The coaxial transseptal device of claim 1, wherein the coaxial guide-wire includes a radial tip at the distal end.

7. The coaxial transseptal device of claim 6, wherein the radial tip is constructed from a radiopaque material.

8. The coaxial transseptal device of claim 1, wherein a flexibility of the shaft of the piercing device increases distally.

9. The coaxial transseptal device of claim 8, wherein the shaft of the piercing device includes a helical cut section on a distal end for increasing the flexibility.

10. The coaxial transseptal device of claim 9, wherein the helical cut section has a pitch that varies along the shaft, whereby the pitch is smaller at a distal end than at a proximal end of the helical cut section.

11. The coaxial transseptal device of claim 9, wherein the helical cut section has a depth that varies along the shaft, whereby the depth is deeper at a distal end than at a proximal end of the helical cut section.

12. The coaxial transseptal device of claim 1, wherein the piercing device includes a hub on the proximal end of the shaft that is adapted to enable the manipulation of the piercing device.

13. The coaxial transseptal device of claim 12 further comprising:
a safety clip configured to attach to the shaft of the piercing device between the hub and the proximal end of the coaxial guide-wire, wherein the safety clip prevents the sharpened portion of the piercing device from extending beyond the distal end of the coaxial guide-wire.

14. The coaxial transseptal device of claim 1, wherein the coaxial guide-wire further comprises:
a removable adapter connected to the proximal end of the coaxial guide-wire and that couples to a pressure monitor for measuring a pressure within a chamber of the heart.

15. A guide-wire comprising:
a tube having proximal and distal ends with a lumen extending therebetween, wherein the flexibility of the tube increases distally; and
a coil surrounding at least the distal end of the tube.

16. The guide-wire of claim 15, wherein the tube includes a helical cut on the distal end that increases the flexibility of the tube.

17. The guide-wire of claim 16, wherein the helical cut includes a pitch that varies along the tube, whereby the pitch is smaller at a distal end than at a proximal end of the helical cut.

18. The guide-wire of claim 16, wherein a direction of the helical cut of the tube opposes a direction of the coil surrounding the tube.

19. The guide-wire of claim 15 further comprising:
a sleeve surrounding the proximal end of the tube and configured to decrease the flexibility of the proximal end of the tube.

20. The guide-wire of claim 15 further comprising:
a removable adapter connected to the proximal end of the tube, the removable adapter coupled to a pressure monitor for measuring a pressure within a chamber of the heart.

21. The guide-wire of claim 15, wherein the lumen of the guide-wire receives a tissue piercing device.

22. A piercing device comprising:
a shaft having a proximal end and a distal end; and
a sharpened portion on the distal end of the shaft, wherein the flexibility of the shaft increases distally.

23. The piercing device of claim 22, wherein the shaft includes a helical cut section on the distal end that increases the flexibility of the distal end of the shaft.

24. The piercing device of claim 23, wherein the helical cut section includes a pitch that varies along the shaft, whereby the pitch is smaller at a distal end than at a proximal end of the helical cut section.

25. The piercing device of claim 23, wherein the helical cut section includes a depth that varies along the shaft, whereby the depth is deeper at a distal end than at a proximal end of the helical cut section.

26. The piercing device of claim 22 further comprising:
a hub attached to the proximal end of the shaft and adapted to enable the manipulation of the piercing device.

27. A method of piercing a tissue within the heart with a coaxial transseptal device comprising a piercing device and a coaxial guide-wire, the piercing device comprising a shaft having a proximal end, a distal end, and having a sharpened portion on the distal end, the coaxial guide-wire having a proximal end and a distal end, wherein a flexibility of the coaxial guide-wire increases distally, and the coaxial guide-wire is configured to receive the piercing device and move relative thereto, the method comprising:
introducing the distal end of the coaxial guide-wire into a superficial blood vessel;
percutaneously directing the distal end of the coaxial guide-wire from the superficial blood vessel to the tissue in a first chamber of the heart;
advancing the sharpened portion of the piercing device distally beyond the distal end of the coaxial guide-wire, thereby puncturing the tissue and entering a second chamber of the heart; and
advancing the distal end of the coaxial guide-wire over the piercing device and into the second chamber, thereby dilating the puncture in the tissue.

28. The method according to claim 27, wherein the superficial blood vessel is a subclavian vein, a jugular vein, a femoral vein, or an iliac vein.

29. The method according to claim 27 further comprising:
advancing a guide catheter to the first chamber before percutaneously directing the distal end of the coaxial guide-wire into the superficial blood vessel.

30. The method according to claim 27, wherein the coaxial guide-wire further includes a removable adapter on the proximal end, the removable adapter being also coupled to a pressure monitor and configured to measure a pressure within the second chamber of the heart.

31. The method according to claim 27, wherein the first chamber is the right atrium, the second chamber is the left atrium, and the tissue is an intra-atrial septum.

32. A method of puncturing a tissue within the heart with a coaxial transseptal device comprising a piercing device and a coaxial guide-wire, the piercing device comprising a shaft having a proximal end, a distal end, and having a sharpened portion on the distal end, the coaxial guide-wire having a proximal end and a distal end, wherein a flexibility of the coaxial guide-wire increases distally and the coaxial guide-wire is configured to receive the piercing device and move relative thereto, the method comprising:
securing the sharpened portion of the piercing device within the coaxial guide-wire;
introducing the distal end of the coaxial guide-wire into a superficial blood vessel;
percutaneously directing the distal end of the coaxial guide-wire from the superficial blood vessel to the tissue in a first chamber of the heart;
releasing the sharpened portion such that the shaft moves relative to the coaxial guide-wire;
advancing the sharpened portion of the piercing device distally beyond the distal end of the coaxial guide-wire, thereby puncturing the tissue and entering a second chamber of the heart;
advancing the distal end of the coaxial guide-wire across the intra-atrial septum and into the second chamber, thereby dilating the puncture in the tissue; and
retracting the piercing device from the coaxial guide-wire.

33. The method according to claim 32, wherein the superficial blood vessel is a subclavian vein, a jugular vein, a femoral vein, an iliac vein.

34. The method according to claim 32 further comprising:
directing a guide catheter into the superficial blood vessel before introducing the distal end of the coaxial guide-wire.

35. The method according to claim 32, wherein the piercing device includes a hub on the proximal end of the shaft to enable the manipulation of the piercing device.

36. The method according to claim 35, wherein a safety clip secures the sharpened portion within the coaxial guide-wire, the safety clip including a clip portion that attaches to the shaft of the piercing device between the hub and the proximal end of the coaxial guide-wire.

37. The method according to claim 32, wherein the distal end of the coaxial guide-wire includes a radial tip constructed from a radiopaque material for in vivo localization.

38. The method according to claim 32, wherein the coaxial guide-wire includes a coil constructed from a radiopaque material for in vivo localization.

39. The method according to claim 32, wherein the distal end of the shaft is constructed from a radiopaque material for in vivo localization.

40. The method according to claim 32, wherein the sharpened portion of the piercing device is constructed from a radiopaque material for in vivo localization.
